(19) United States
(12) Patent Application Publication
(10) Pub. No.: US 2022/0193336 A1
(43) Pub. Date: Jun. 23, 2022

(54) SYNCHRONIZATION OF PATIENT ASSOCIATION DATA ACROSS A HEALTHCARE ORGANIZATION NETWORK

(71) Applicant: CareFusion 303, Inc., San Diego, CA (US)

(72) Inventors: Michael K. WORKMAN, Carlsbad, CA (US); Lisa DIGGETT, Overland Park, KS (US)

(21) Appl. No.: 17/555,287

(22) Filed: Dec. 17, 2021

Related U.S. Application Data
(60) Provisional application No. 63/127,970, filed on Dec. 18, 2020.
(60) Provisional application No. 63/171,056, filed on Apr. 5, 2021.

(51) Int. Cl. A61M 5/168; A61M 5/142; G16H 20/17; G16H 10/60

(52) U.S. Cl. CPC A61M 5/168; A61M 5/142; G16H 20/17; A61M 2005/14208; A61M 2205/3553; A61M 2205/584; G16H 10/60

(57) ABSTRACT

An infusion device includes a pump, and a control unit. The control unit is configured to provide an infusion of a medication to a current patient, send infusion information, during the infusion, to a remote records system, receive, from the records system, a confirmation of the infusion information including an association between the infusion and the patient or the pump, receive an indication of a change to the infusion, compare the change with the association information received from the system, determine, based on the comparing, that the association received from the system no longer applies to the infusion, update the representation of the status displayed on the display screen to indicate that the association information received from the system no longer applies to the infusion, and send a message to the system indicating that the infusion device has determined that the association information no longer applies to the changed infusion.

### CROSS-REFERENCE TO RELATED APPLICATIONS

This application claims the benefit of U.S. Provisional Application Ser. No. 63/127,970, entitled “SYNCHRONIZATION OF PATIENT ASSOCIATION DATA ACROSS A HEALTHCARE ORGANIZATION NETWORK,” filed on Dec. 18, 2020, and claims the benefit of U.S. Provisional Application Ser. No. 63/171,056, entitled “SYNCHRONIZATION OF PATIENT ASSOCIATION DATA ACROSS A HEALTHCARE ORGANIZATION NETWORK,” filed on Apr. 5, 2021, the entirety of each of which is incorporated herein by reference.

### BACKGROUND

The present disclosure generally relates to systems and methods of identifying, associating and dissociating medical equipment with patients.

A hospital or other care giving institution records and tracks the treatment given to patients. One method commonly used is the recording and maintaining of an electronic medication administration record, typically called an eMAR, that contains details of each medication administration given to a patient in the institution, including the medication administered as well as the device used to administer the medication. While here and below, reference is made to storing records in an eMAR, other health information management systems (e.g., patient data management systems (PDMSs)) may create or store records of medication administration for a patient. The eMAR is a document that, to be useful, must be accurate, and contain a complete record of a patient's treatment.

A server connected to a medical device may then keep a record of the medication administration in its own memory or database, or may also communicate the relevant information to the hospital system, such as an eMAR system, for recordation in the patient's eMAR. The eMAR system may include a medication administration record database that stores the electronic medication administration (eMAR) records of patients. It will immediately be apparent to those skilled in the art that the medication order number is central to maintaining an accurate and complete record of medications delivered to a patient. All medication administrations are identified by a unique medication order identification number, which is then associated with a patient.

One problem that arises, however, is that changes may be made to an infusion regimen after the initial start of the infusion is recorded in the eMAR. In some situations, such as in an emergency room, a medical device may quickly be switched from one patient to another, or medications quickly provided to a patient without a formal order for the medications. Moreover, device modules may be disconnected from a medical device and moved to a different medical device for the benefit of another patient. Such changes should be recorded in the patient's eMAR to ensure a complete record of the medications given to the patient. However, prior systems have not been capable of communicating changes in the field to the eMAR system. In this regard, the server and/or eMAR system may continue to store a current association between the device module and the first patient, but may not be notified of the use by the second patient.

Moreover, in such cases, there may be no pharmacy generated medication order identification number assigned, and the disconnection from the first patient may not be accurately communicated to and stored in the eMAR system.

Because the eMAR is often a record of truth for clinical decision making and automated medical device control, these technical issues with association and disassociation of devices and patients can impact life critical decisions.

### SUMMARY

In order to improve the safety of the patient while simultaneously eliminating at least some of the time-consuming steps of manually scanning barcodes to identify one or more of the patient, the caregiver, the medication, or the medical device, it is advantageous to provide an association of a patient with the medical device and a medication or other medical substance that are physically proximate to them. Moreover, as medical devices such as infusion pumps are incorporated into a hospital system, the need to efficiently and accurately document care provided by the devices becomes an important factor. One area of importance is to ensure the underlying record of events accurately reflect medication or other therapies provided to a patient. For example, consider the situation where an infusion pump is associated with a first drug being infused to a first patient. That infusion device is disconnected and used to provide a different drug to a second, different, patient. The systems involved to manage the care for the first and second patients may not be aware that the same device is being used and inaccurately attribute administration of the first or second drug with the wrong patient.

The subject technology described herein addresses the problems with existing methods while providing efficiency and accuracy desired by modern day medical environments and organizations. In this regard, the subject technology includes an infusion device comprising a pump and a control unit. The control unit is configured to provide, using the pump, an intravenous infusion of a medication to a current patient, display on a display screen, while the infusion is being provided by the pump, a representation of a status of the intravenous infusion, send, during the infusion, to a remote records system, infusion information including a patient identifier and order identifier for the infusion currently being provided by the pump, receive, from the remote records system, a confirmation of the infusion information including association information representative of an association between the infusion and the current patient or the pump, receive, at the infusion device, an indication of a change to the infusion currently being provided by the pump, compare the change with the association information received from the remote records system, determine, based on comparing the change with the association information received from the remote records system, that the association received from the remote records system no longer applies to the changed infusion, update the representation of the status displayed on the display screen to indicate that the association information received from the remote records system no longer applies to the intravenous infusion, and send a message to the remote records system indicating that the infusion device has determined that the association information no longer applies to the changed infusion. Other aspects include corresponding systems, apparatuses, methods, and computer program products for implementation of the foregoing features.

The methods and features may be implemented in whole or in part by a medical device, such as an infusion pump or an electronic health information system such as an EMR or device management server.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE FIGURES

The accompanying drawings, which are included to provide further understanding and are incorporated in and constitute a part of this specification, illustrate disclosed implementations and together with the description serve to explain the principles of the disclosed implementations. In the drawings:

FIG. 1 depicts an example flow diagram of data associations between a patient, one or more pharmacy orders, and an infusion pump, according to various aspects of the subject technology.

FIG. 2 depicts an example flow diagram of unassociated data for a patient, one or more pharmacy orders, and an infusion pump, according to various aspects of the subject technology.

FIGS. 3A and 3B are data flow diagrams depicting example data flow from an infusion device to a healthcare information system for associating a patient identifier, according to various aspects of the subject technology.

FIGS. 4A and 4B are data flow diagrams depicting example data flow from an infusion device to a healthcare information system for associating a device identifier, according to various aspects of the subject technology.

FIG. 5 depicts an example data flow diagram for sharing associations between an infusion device and a healthcare information system, according to various aspects of the subject technology.

FIG. 6 depicts an example diagram of an institutional patient care system 100 of a healthcare organization, according to aspects of the subject technology.

FIG. 7 depicts an example process flow for automatically associating or disassociating an infusion and a patient or a medication, according to aspects of the subject technology.

FIG. 8 depicts an example process for an infusion device-initiated association or disassociation of an infusion and a patient or a medication, according to aspects of the subject technology.

FIG. 9 depicts an example process flow for a server initiated association or disassociation of an infusion and a patient or a medication, according to aspects of the subject technology.

FIG. 10 is a conceptual diagram illustrating an example electronic system 400 for automatically associating or disassociating an infusion and a patient or a medication, according to aspects of the subject technology.

### DESCRIPTION

The disclosed methods and system provide for the the synchronization of association and disassociation healthcare system elements across a healthcare organization network, and ensures the proper associations between the elements are recorded at the eMAR system. These elements include one or more of a patient, a medical device, a medication or medical substance, and any nurse, doctor, or other caregiver. Once these items are associated, one or more of the elements may be configured according to the association with other elements, privileges may be granted based on the identification of the caregiver present in the physical area, and records kept of actions and events that occur. Moreover, records of the association are updated and/or confirmed as being accurate across the systems of the healthcare organization, thereby ensuring that the proper patient is receiving therapy from the proper device and/or medication.

In the following detailed description, numerous specific details are set forth to provide a full understanding of the present disclosure. It will be apparent, however, to one ordinarily skilled in the art that implementations of the present disclosure may be practiced without some of the specific details. In other instances, well-known structures and techniques have not been shown in detail so as not to obscure the disclosure.

The method and system disclosed herein are presented in terms of the administration of a medication as an IV fluid using an IV pump to a patient in a hospital. The method and system, however, are equally applicable to other medical settings such as an outpatient clinic and to nonmedical applications where it is desirable to associate various elements based on a common presence within a defined physical space. Nothing in this disclosure should be interpreted, unless specifically stated as such, to limit the application of any method or system disclosed herein to a medical or hospital environment.

It is advantageous to have a care management system that combines all the various medication order and administration services of a healthcare facility into an integrated, automated system that checks and documents the delivery of therapeutic and other drugs to the patient. Such a system would prevent administering an inappropriate medication to a patient by checking the medication against a database of known allergic reactions and/or side-effects of the drug against the patient's medical history. The integrated system should also provide doctors, nurses, and other care-givers with updated patient information at the bedside, notify the facility's pharmacy when an additional drug is required, or when a scheduled treatment is running behind schedule, and automatically update the facility's accounting database each time a medication or other care is given.

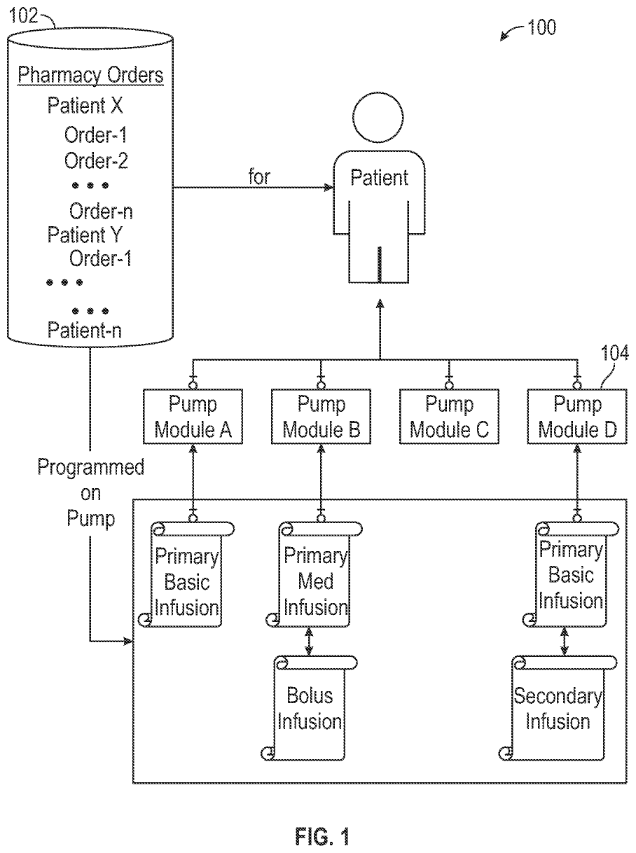

FIG. 1 depicts an example flow diagram 100 of data associations between a patient, one or more pharmacy orders, and an infusion pump, according to various aspects of the subject technology. According to various aspects, medical element association, as described herein, includes the process of correlating a patient, one or more orders, and infusion information from an infusion device. A medical information server and/or eMAR system, acting as a medical information “consumer” 102, is responsible for recording and maintaining patient and order information, as well as maintaining associations between that information for all the patients and infusion devices utilized across a hospital network. In this regard, each infusion device, when connected to the network, reports infusion data related to the progress of the infusion(s) it performs.

In some implementations, an infusion device, acting as a “reporter” 104, is configured to support auto documentation by suppling event data to the consumer(s). The auto documentation functionality is typically owned and performed by the consumer device(s). In many healthcare environments, the consumer may be responsible for establishing link between the pump infusion data and the consumer's patient and order information within the consumer application (e.g. in an eMAR system).

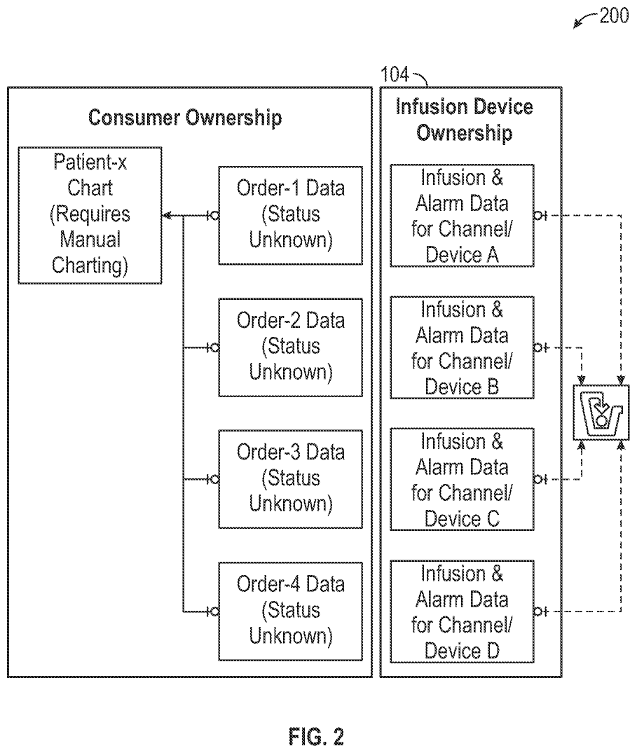

FIG. 2 depicts an example flow diagram 200 of unassociated data for a patient, one or more pharmacy orders, and an infusion pump, according to various aspects of the subject technology. In the depicted example, no association between the consumer 102 and reporter 104 is maintained or recorded. Without an association the Infusion data is unable to flow into the patient chart.

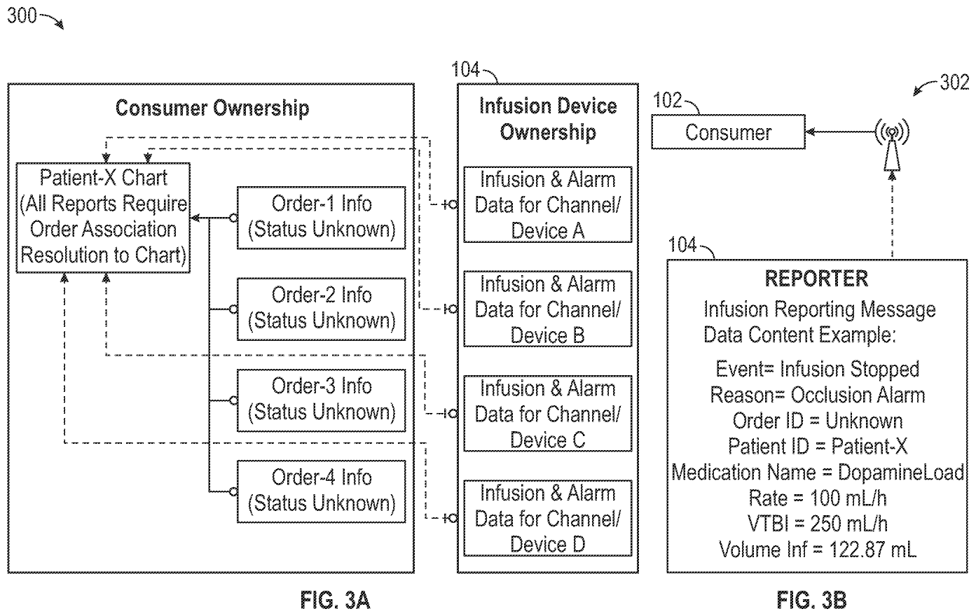

FIGS. 3A and 3B depict example data flow 300, 302 from an infusion device to a healthcare information system for associating a patient identifier, according to various aspects of the subject technology. An infusion device (including, e.g., a pump or pump module) (acting as a reporter 104) may be stationary, and may generally plug into a device rack or patient care unit, which is assigned to patient bed or room. A location identifier (ID) or proxy ID is published by the infusion device within the infusion data sent by the device over the hospital information network. The ID can then be mapped the consumer 102 (e.g. an eMAR system) to a patient ID. Such assignment has several advantages including the location ID being easily automatable, there is no need for additional hardware scanners or workflows, and patient to infusion data association and disassociation can be automated by the reporter (e.g. the infusion device).

According to some implementations, a mobile device may be implemented in the association workflow. In this regard, a patient identifier is scanned (e.g., using a barcode or RFID scanner) or programmed on the reporter device as input. Association and disassociation events may be published by the mobile device, as well as the reporting of the patient identifier with the infusion data to associate infusion data to patient. U.S. Pat. No. 10,275,571 further describes using a mobile device for interacting with infusion data reporting, the entirety of which is incorporated by reference herein for that and related purposes.

The configuration shown in FIGS. 3A and 3B provide features to create an association between the infusion device and a patient in the consumer system. However, the configuration may fail to provide adequate association. For example, the association in FIG. 3A is predicated on accurate reporting of the patient identifier. In the case where an infusion pump is quickly reprogrammed for a new patient, the patient identifier may be used to associate events to a different patient's record. As another example, if a newly admitted patient (e.g., in the emergency room) does not yet have a patient identifier, the infusion events may be reported to the consumer but the consumer may have no mechanism to attribute the events to a specific record. A final association challenge may arise based on the nature of the medical device. Some infusion pumps, for example, are modular infusion devices whereby multiple pumps may be controlled by a central patient care unit (PCU). For such devices, it may be desirable to associate events for specific modules to ensure that volume of a first drug infused by one module is not improperly counted as a volume of infused by a second, different module.

Another consideration with the configuration shown in FIGS. 3A and 3B is the solution may be predicated on accurate location identification. When devices are attached to fixed positions (e.g., network location), the location can serve as an accurate association indicator. However, if the medical device is mobile (e.g., wirelessly connected to the hospital network), the medical device location can vary and, in some instances, have no relationship to a nearby patient.

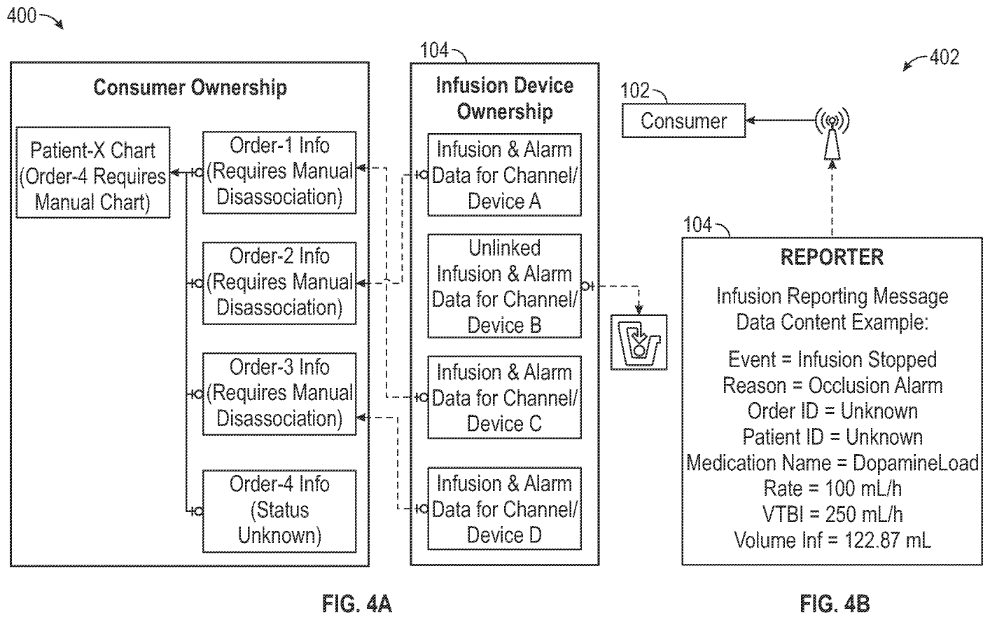

FIGS. 4A and 4B are data flow diagrams 400, 402 depicting example data flow from an infusion device to a healthcare information system for associating a device identifier, according to various aspects of the subject technology. In some implementations, the patient is associated with an order and to the infusion data through the device ID of the infusion device. This may take place on the consumer side 102 (e.g. eMAR) and may not be visible to the reporter 104 (e.g. the infusion device).

According to one example, a device ID is scanned as part of input for the consumer 102 (e.g. eMAR application). The device ID is selected from a list of possible devices known to the consumer application. A complete association may then be made between the infusion data provided by the infusion device and patient and order information, allowing infusion data to flow into the patient's chart immediately for a given order. This may have minimal impact on clinical workflow within the consumer application. However, continued manual actions by a user may take place to impact the synchronization status between the reporter and infusion device. For example, a clinician may need to monitor data flowing from the medical device and manually like the data with a patient record. As another example, if the medical device completes an infusion for a first patient and the device is immediately used for a second patient. If the clinician forgets to re-associate the device with the second patient, the second patient's data will be recorded in the first patient's record. Such associations should be made for each infusion device on the remote consumer system. In some systems, manual synchronization should be consistent to avoid incomplete documentation for a patient chart.

As with the configuration shown in FIG. 3A, the configuration shown in FIGS. 4A and 4B provide a level of synchronization between the reporter and consumer. One key factor in the synchronization is proper linking of a device ID to a patient. As discussed, if the device is quickly repurposed or assigned to a patient without an identifier, the events reported by the infusion device may be unassociated. Another condition which may arise in systems that rely on patient identifier is how the disassociation occurs when a patient is discharged. In some cases, a pump may be properly associated with a patient using the device ID the consumer links to a patient identifier. After the patient is discharged from the care facility, the same pump may be used to initiate a new infusion to a different patient. Either on error or on purpose (e.g., to divert drugs), a user may initiate an infusion using the discharged patient's identifier. Only after the infusion reports an event would the consumer be able to detect that this infusion should not occur. However, if the systems were synchronized, the infusion may be prevented from being initiated until associated with a current patient.

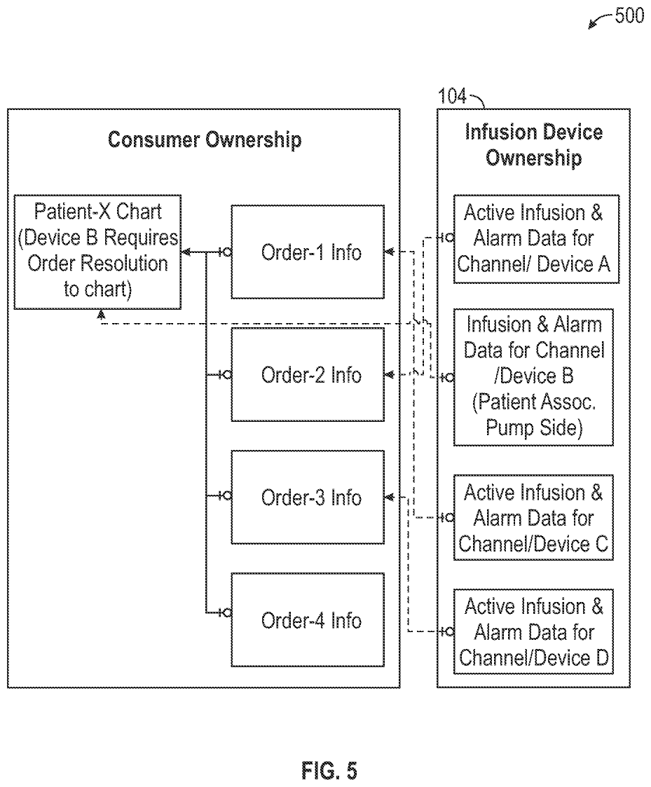

FIG. 5 depicts an example data flow diagram 500 for sharing associations between an infusion device and a healthcare information system, according to various aspects of the subject technology.

According to various implementations, a patient identifier is supplemented with a device ID of an infusion device providing an intravenous (IV) infusion to the patient. This association can occur at either the infusion device (as a reporter 104) or a remote records system (operating as the consumer 102). One or more messages are sent between the systems to maintain synchronization of the patient and device IDs.

The subject technology provides a patient, order, and infusion association interoperability model which creates a protocol for coupling infusion reporters 104 and infusion data consumers 102 with regard to infusion data flowing into orders for patient charts. The subject protocol between the infusion reporter 104 and infusion data consumer further automates the patient order association and disassociation to infusion data. In this regard, the subject technology creates a feedback to the infusion reporter 104 of an association made to provide a more indication to the user that association was successful, as well as allows improved and more accurate disassociation of patient order to infusion data; which prevents errors in the patient chart.

According to various implementations, if a reporter device ID is associated or disassociated to a patient and/or order information within the consumer application 102 (e.g. eMAR system) then a corresponding association or disassociation message may be sent to the reporting device ID. The message may include the patient and order identifiers associated or disassociated.

In some implementations, the infusion device may provide a visual indication (on its display screen) that it was remotely associated or disassociated such as flash its lights, display an icon, audio, or otherwise providing a confirmation that the correct pump was synchronized. And, if a new patient ID is entered or an existing patient ID is changed or cleared on the infusion device, then a corresponding association or disassociation message(s) is sent to the consumer system 102.

Accordingly, complete associations may be made between infusion data provided by the infusion device and patient and order information, allowing infusion data to flow into chart immediately for a given order. In this regard, there is minimal workflow impact on clinical workflow within the consumer application 102, as associations and disassociations are automatically synchronized between consumer 102 and reporter systems 104, reducing the manual steps to disassociate, and thus reducing charting errors, particularly useful for disassociations. Because associations may be made on the reporter device, the chances of incomplete documentation for patient chart are reduced or obviated.

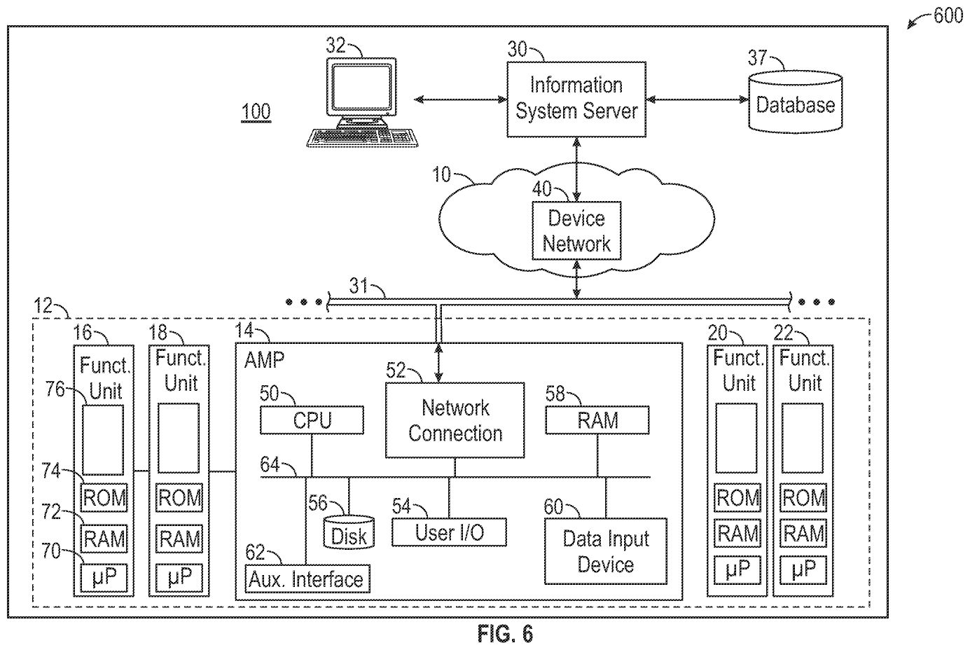

FIG. 6 depicts an example diagram of an institutional patient care system 600 of a healthcare organization, according to aspects of the subject technology. In FIG. 6, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. As described previously, PCU/PCD 12 (and/or the infusion pump) functions as the previously described reporter 104.

Each device 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care devices 12 (and other devices) communicate in accordance with normal operations.

Additionally, institutional patient care system 600 may incorporate a separate information system server 30. According to various implementations, server 30 and/or database 37 may incorporate, function as, or include a remote records system configured to store electronic medication administration records (eMAR) for patients admitted or cared for within the hospital organization. In this regard, according to various implementations server 30 and/or the eMAR system may function as the previously described consumer 102. Server 30 may further communicate with or include a database 37. Server 30 and/or database 37 may also store associations between patient identifiers, identifiers for infusion devices, as well as patient order information for the patients, and receive and record corresponding infusion data received from the infusion devices in real time for those associations. Although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistances, mobile devices such as laptops, tablet computers, augmented reality devices, or smartphones, configured with software for communications with information system server 30 via network 10.

Patient care device 12 comprises a system for providing patient care, such as that described in U.S. Pat. No. 5,713,856 to Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control unit 14, also referred to as interface unit 14 or docking station, connected to one or more functional modules 16, 18, 20, 22. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1 to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with controller 14, or any other system on the network, using suitable programming and communication protocols.

Network connection 52 may be a wired or wireless connection, such as by Ethernet, WiFi, BLUETOOTH, an integrated service digital network (ISDN) connection, a digital subscriber line (DSL) modem or a cable modem. Any direct or indirect network connection may be used, including, but not limited to a telephone modern, an MIB system, an RS232 interface, an auxiliary interface, an optical link, an infrared link, a radio frequency link, a microwave link or a WLANS connection or other wireless connection.

Functional modules 16, 18, 20, 22 are any devices for providing care to a patient or for monitoring patient condition. As shown in FIG. 6, at least one of functional modules 16, 18, 20, 22 may be an infusion pump module such as an intravenous infusion pump for delivering medication or other fluid to a patient. For the purposes of this discussion, functional module 16 is an infusion pump module. Each of functional modules 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor or an intracranial pressure monitor or the like. Functional module 18, 20 and/or 22 may be a printer, scanner, bar code reader or any other peripheral input, output or input/output device.

Each functional module 16, 18, 20, 22 communicates directly or indirectly with interface unit 14, with interface unit 14 providing overall monitoring and control of device 12. Functional modules 16, 18, 20, 22 may be connected physically and electronically in serial fashion to one or both ends of interface unit 14 as shown in FIG. 6. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 6, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 16.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection 54 (as shown in FIG. 1), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in HIS server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices, and maintain open communication.

With further reference to FIG. 6, an infusion device, as used herein, may be a patient care device 12, interface control unit 14, or a module 16, 18, 20, 22. According to various implementations, the infusion device includes a pump and a control unit. The control unit 14 is configured to provide, using the pump, an intravenous infusion of a medication to a current patient, and display on a display screen, while the infusion is being provided by the pump, a representation of a status of the intravenous infusion. The representation of the status may include, for example, a single colored light emanating from an LED affixed to the control unit, pump, or infusion device generally, or may be an element displayed on the display screen. In some implementations, the colored light bay be a backlight or lighted outline around content in a display screen.

Control unit 14 is configured to send, during the infusion, to a remote records system 30, infusion information including a patient identifier and order identifier for the infusion currently being provided by the pump. For example, a caregiver may input that a particular patient is receiving a certain dose of acyclovir, and the patient ID, drug ID, and dosage information may be sent to system 30 along with the device ID of the infusion device. Records system 30 then sends to control unit 14 (for which control unit 14 is configured to then receive), a confirmation of the infusion information including association information representative of an association between the infusion and the current patient or the pump. This association confirms the eMAR system has accepted that the patient is to receive the acyclovir in the dosage given. For example, the eMAR system may indicate to the pump that it is infusing on patient X for order Y.

Control unit 14 may receive, for example from interface device 54 at the infusion device, an indication of a change to the infusion currently being provided by the pump. For example, a caregiver may input parameters into the infusion device indicative of a new medication, a new patient, or a new order for the current patient. Control unit 14 compares the change with the association information received from the remote records system, and determines, based on comparing the change with the association information received from the remote records system, whether the association received from the remote records system no longer applies to the changed infusion. Control unit 14 then proceeds to update the representation of the status displayed on the display screen to indicate that the association information received from the remote records system no longer applies to the intravenous infusion. This provides the caregiver the opportunity to change any mistake that was made or to confirm the change.

Upon confirmation of the change by the caregiver, control unit 14 is configured to send a message to the remote records system 30 indicating that the infusion device has determined that the association information no longer applies to the changed infusion. For example, a message is sent to the eMAR system that the order ID that system 30 believes is being infused by the infusion device is no longer valid. The system 30 may then choose to take an action, depending on its predetermined program implementation. For example, if the patient changed at the infusion device, system 30 may break the association between all modules 16, 18, 20, 22 associated with the infusion device and the current patient. If the drug changed, system 30 may stop accumulating the current drug (e.g. acyclovir) and begin recording an accumulation of the new drug. In some implementations, on the patient or drug being changed, system 30 may inform control unit 14 to terminate the related infusions or request confirmation as to whether the infusions should continue.

In some implementations, if the medication changed, system 30 may also start to buffer infusion data pertaining to the new medication until the changed infusion is confirmed at the remote records system 30, for example by an authorized care administrator at terminal 32. On the changed infusion being confirmed, the remote records system 30 is configured to create an association between the changed infusion and the current patient or the infusion device, and may start recording data provided by the infusion device for the changed infusion in association with the current patient or the infusion device.

According to various implementations, whether system 30 automatically acts upon a change notification from the infusion device may depend on whether the change notification is representative of a hard change or a soft change. A hard change may, for example, indicate a change to a medication not ordered for a particular patient, a change in dosage over a threshold amount, or an assignment of the infusion device to a new patient. In these situations, system 30 may send an alert to a mobile device associated with an authorized administrative caregiver or the patient's primary caregiver, or may pause or terminate one or more infusions as described above, or request confirmation before such termination, and/or perform an automatic dissociation and reassociation in accordance with the information received from the infusion device. A soft change may, for example, include receiving an indication that a new infusion set was applied to the current infusion, the volume of the order changed, or the current infusion is being titrated. In these situations, system 30 may merely record the change and send the alert.

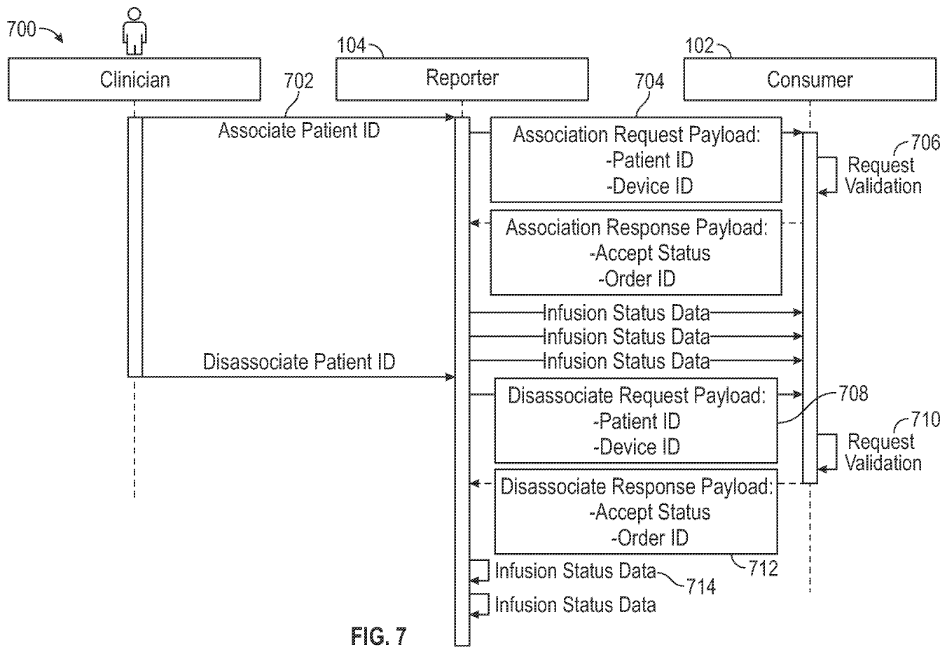

FIG. 7 depicts an example process flow 700 for automatically associating or disassociating an infusion and a patient or a medication, according to aspects of the subject technology. For explanatory purposes, the various blocks of example process 700 are described herein with reference to FIGS. 1 to 6, and the components and/or processes described herein.

In the depicted example, a clinician sets up an infusion device to delivery an intravenous infusion of a medication to a current patient, and the infusion device provides, using a pump, the infusion of the medication to the patient. As part of this process, the clinician programs the infusion device (e.g. control unit 14), functioning as reporter 104, with a patient ID and a medication ID (702). The infusion device begins the infusion and at the same time, or before initiating the infusion, sends an association (or disassociation) request to system 30, functioning as consumer 102 (704). The request may include infusion information, which the patient ID and medication/order ID for the infusion currently being provided by the device. System 30 performs a validation of the data received from the infusion device and sends a confirmation of the infusion information including association information representative of an association between the infusion and the current patient (706). The infusion continues (or begins), and infusion device sends status information to system 30 during the infusion. Status information may also be displayed by the infusion device.

At some point, the infusion device may receive an indication of a change to the infusion currently being provided to the patient. The infusion device is configured to compare the change with the association information received from the remote records system and determine, based on comparing the change with the association information received from the remote records system, that the association received from the remote records system no longer applies to the changed infusion. When the association no longer applies, infusion device is configured to send a disassociation request to system 30 (708). In this regard, system 30 receiving the indication that the infusion device has determined that the association information no longer applies to the infusion or the current patient.

In connection with the disassociation request, system 30 may receive, from the infusion device, new association information including a new patient identifier and/or a new medication (e.g. for the current or new patient). System 30 may automatically initiate creation of a new association between the infusion device and the new patient or the new medication based on the new association information received from the infusion device. As part of this process, system 30 may perform a second validation of the new information received from the infusion device (710). For example, system 30 may send a message to an authorized administrator to validate that the disassociation occurred or to confirm the new association. Infusion device may display on the display screen an indication that system 30 is not synchronized with the changed infusion to include the association between the changed infusion and the current patient or the infusion device, and prompt the clinician for a confirmation to proceed with the changed infusion. System 30 may then automatically disassociate the current patient or the current infusion from the infusion device, and send confirmation of the new association back to the infusion device (712). The infusion device may then continue with the infusion based on the new association, displaying a status of the infusion in real time to the clinician (714).

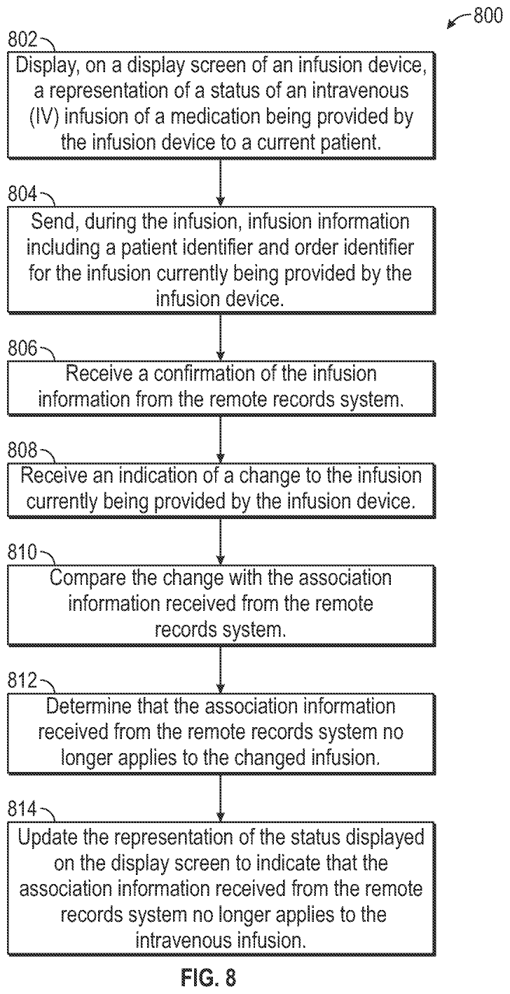

FIG. 8 depicts an example process 800 for an infusion device-initiated association or disassociation of an infusion and a patient or a medication, according to aspects of the subject technology. For explanatory purposes, the various blocks of example process 800 are described herein with reference to FIGS. 1 to 7, and the components and/or processes described herein. The one or more of the blocks of process 800 may be implemented, for example, by one or more computing devices including, for example, medical device 12. In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 800 are described as occurring in serial, or linearly. However, multiple blocks of example process 800 may occur in parallel. In addition, the blocks of example process 800 need not be performed in the order shown and/or one or more of the blocks of example process 800 need not be performed.

In the depicted example, an infusion device displays, on a display screen of the infusion device, a representation of a status of an intravenous infusion of a medication being provided by the infusion device to a current patient (802). The representation of the status may be implemented as a single colored light such as an LED, or a graphical element in a graphical interface of a display screen. The infusion device (e.g. control unit 14 or a module 16, 18, 20, 22) sends, during the infusion, infusion information including a patient identifier and order identifier for the infusion currently being provided by the infusion device (804). In some implementations, the control unit 14 or a module associated therewith may send an association message which causes all modules for the control unit 14 to be associated according to the association message. For example, if a docking station includes three pumps and a first pump sends or receives a message to associate with a patient, the other two pumps may also associate with the same patient. A confirmation of the infusion information is received from the remote records system 30 (806). According to various examples, the confirmation includes association information representative of an association between the infusion and the current patient or the infusion device. In the docking station example, each pump in the docking station may receive an association confirmation message including the specific order identifier to be infused by the respective pump.

The infusion device receives an indication of a change to the infusion currently being provided by the infusion device (808). The indication may include receiving parameters corresponding to a new medication or new patient (or new order) at an input interface of the infusion device, for example entered by a clinician. The infusion device compares the change with the association information received from the remote records system (810) and determines, based on comparing the change with the association information received from the remote records system, that the association information received from the remote records system no longer applies to the changed infusion (812). The comparison may include assessing correspondence between a prior value and the new value. Examples of parameters that may be used to assess association status include: patient weight, patient name, patient identifier, drug identifier, drug concentration, drug container, volume to be infused, dosing information, control unit identifier, or the like.

The infusion device updates the representation of the status displayed on the display screen to indicate that the association information received from the remote records system no longer applies to the intravenous infusion (814). This may include changing a color of the single colored light from a first color to a second color. According to some implementations, green may mean the association is current and verified, while red may indicate that the association is not verified and does not match. A third, yellow color may be reserved for situations in which a soft change occurred. Concurrently or thereafter, infusion device sends a message to remote records system 30 indicating that the infusion device has determined that the association information no longer applies to the changed infusion (816).

According to various implementations, a change to the infusion currently being provided by the infusion device may include changing the medication to a new medication. In this regard, the association information received from the remote records system may include an indication that a record for the current patient stored at the remote records system has not been updated with an order for the new medication. In some implementations, the message sent to system 300 may instruct the system 30 to stop recording infusion data pertaining to the medication and to start recording infusion data pertaining to the new medication. The instruction may not be explicit. For example, the indication of the change may be interpreted by system 30 as an instruction.

According to various implementations, a change to the infusion currently being provided by the infusion device may include changing the current patient to a new patient. In this regard, the association information received from the remote records system may include an indication that the infusion device has not been assigned to the new patient at the remote records system. In some implementations, the message sent to system 300 may instruct the system 30 to disassociate the current patient from the infusion device. The instruction may not be explicit. For example, the indication of the change may be interpreted by system 30 as an instruction.

Additionally, or in the alternative, the message may instruct the remote records system 30 to buffer infusion data pertaining to the new medication until the remote records system until the changed infusion is confirmed at the remote records system. Again, the instruction may not be explicit. On the changed infusion being confirmed at remote records system 30, system 30 may create an association between the changed infusion and the current patient or the infusion device, and begin recording data provided by the infusion device for the changed infusion in association with the current patient or the infusion device.

As described previously, when a change is detected, infusion device may display on the display screen an indication that the remote records system is not synchronized with the changed infusion to include the association between the changed infusion and the current patient or the infusion device, and prompt for a confirmation to proceed with the changed infusion. Infusion device may then adjust infusion parameters on the infusion device to implement the change to the infusion when the confirmation is received (e.g. in accordance with entered parameters). Infusion device may be configured to terminate (or pause) the infusion when the confirmation is not received. Additionally, other modules associated with the infusion device may be providing infusions to the same patient. When a confirmation is not received, the infusion device may terminate the other infusions.

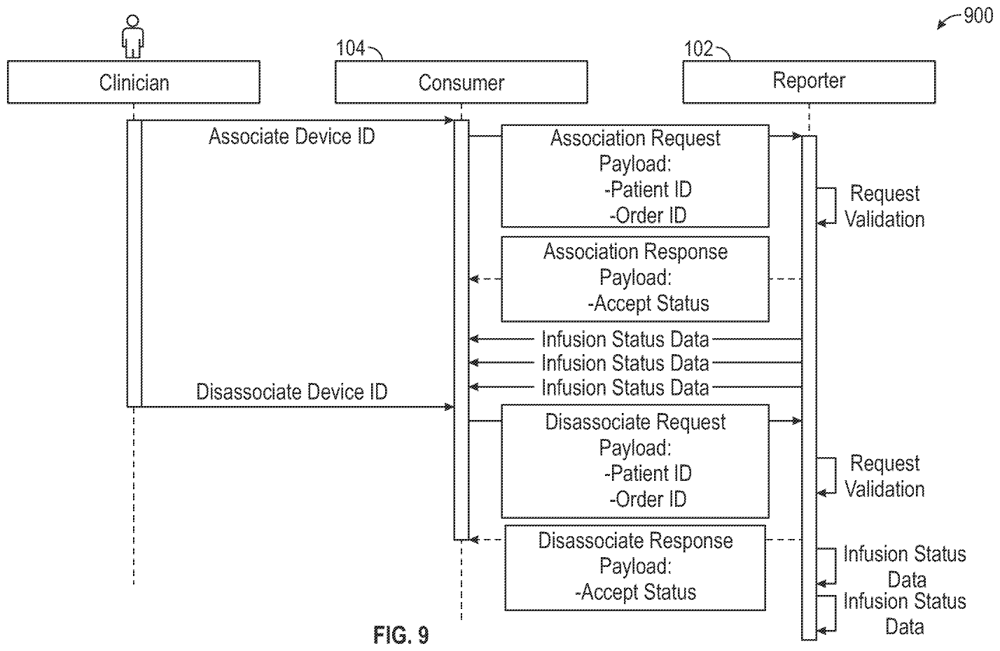

FIG. 9 depicts an example process flow 900 for a server initiated association or disassociation of an infusion and a patient or a medication, according to aspects of the subject technology. The steps of FIG. 9 are similar or identical to the steps of FIGS. 7 and 8 with the exception the clinician programs the association or disassociation, including a patient ID and a medication ID at the server, and the server sends the association or disassociation requests to the infusion device and the infusion device performs the validation and responds with the confirmation. The validation can be performed by the clinician at the patient's bedside or at an associated nursing station or mobile device. The validation may be performed using one or more input devices in communication with the reporter 102 such as a scanner, a user interface, or a sensor. For example, the association request may include an identifier for the clinician requesting the association. This identifier may be provided or associated with a user logging into the reporter 102 device. Validation may include confirming correspondence of the identity of the requestor and the user of the infusion device. In some implementations, the validation may include validating the source or content of the request such as by using a shared secret, encryption/decryption, message hash, or other system for proving validity between the reporter 102 and the consumer 104.

As shown in FIG. 9, after the disassociate request is accepted, new infusion status data may be generated. In some implementations, the reporter 102 may buffer the infusion status data until a new association is confirmed. Once confirmed, the reporter 102 transmit the buffered events to the consumer 104. In some implementations, the infusion status data may include a flag or other value indicating whether the data was buffered (e.g., due to non-association) or transmitted without buffering. In some implementations, the data may be sent to the EMR/PDMS, which can decide to display the data (or portion thereof) with an indication that the data has not yet been validated.

Many of the above-described examples of FIGS. 7, 8, and 9, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

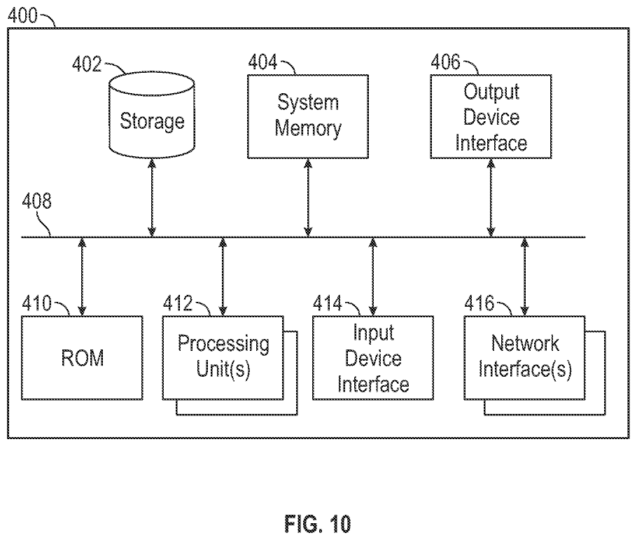

FIG. 10 is a conceptual diagram illustrating an example electronic system 400 for automatically associating or disassociating an infusion and a patient or a medication, according to aspects of the subject technology. Electronic system 400 may be a computing device for execution of software associated with one or more portions or steps of process 400, or components and processes provided by FIGS. 1-9, including but not limited to information system server 30, production server 204, computing hardware within patient care device 12, or terminal device 37. Electronic system 400 may be representative, in combination with the disclosure regarding FIGS. 1-9. In this regard, electronic system 400 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 400 may include various types of computer readable media and interfaces for various other types of computer readable media. In the depicted example, electronic system 400 includes a bus 408, processing unit(s) 412, a system memory 404, a read-only memory (ROM) 410, a permanent storage device 402, an input device interface 614, an output device interface 406, and one or more network interfaces 416. In some implementations, electronic system 400 may include or be integrated with other computing devices or circuitry for operation of the various components and processes previously described.

Bus 408 collectively represents all system, peripheral, and chipset buses that communicatively connect the numerous internal devices of electronic system 400. For instance, bus 408 communicatively connects processing unit(s) 412 with ROM 410, system memory 404, and permanent storage device 402.

From these various memory units, processing unit(s) 412 retrieves instructions to execute and data to process in order to execute the processes of the subject disclosure. The processing unit(s) can be a single processor or a multi-core processor in different implementations.

ROM 410 stores static data and instructions that are needed by processing unit(s) 412 and other modules of the electronic system. Permanent storage device 402, on the other hand, is a read-and-write memory device. This device is a non-volatile memory unit that stores instructions and data even when electronic system 400 is off. Some implementations of the subject disclosure use a mass-storage device (such as a magnetic or optical disk and its corresponding disk drive) as permanent storage device 402.

Other implementations use a removable storage device (such as a floppy disk, flash drive, and its corresponding disk drive) as permanent storage device 402. Like permanent storage device 402, system memory 404 is a read-and-write memory device. However, unlike storage device 402, system memory 404 is a volatile read-and-write memory, such a random access memory. System memory 404 stores some of the instructions and data that the processor needs at runtime. In some implementations, the processes of the subject disclosure are stored in system memory 404, permanent storage device 402, and/or ROM 410. From these various memory units, processing unit(s) 412 retrieves instructions to execute and data to process in order to execute the processes of some implementations.

Bus 408 also connects to input and output device interfaces 414 and 406. Input device interface 414 enables the user to communicate information and select commands to the electronic system. Input devices used with input device interface 414 include, e.g., alphanumeric keyboards and pointing devices (also called “cursor control devices”). Output device interfaces 406 enables, e.g., the display of images generated by the electronic system 400. Output devices used with output device interface 406 include, e.g., printers and display devices, such as cathode ray tubes (CRT) or liquid crystal displays (LCD). Some implementations include devices such as a touchscreen that functions as both input and output devices.

Also, as shown in FIG. 4, bus 408 also couples electronic system 400 to a network (not shown) through network interfaces 416. Network interfaces 416 may include, e.g., a wireless access point (e.g., Bluetooth or WiFi) or radio circuitry for connecting to a wireless access point. Network interfaces 416 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 400 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user: e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

In any implementation disclosed herein, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, virtually (e.g., different network addresses) and/or physically. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

The previous description is provided to enable a person of ordinary skill in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the terms “a set” and “some” refer to one or more. Pronouns in the masculine (e.g., his) include the feminine and neuter gender (e.g., her and its) and vice versa. Headings and subheadings, if any, are used for convenience only and do not limit the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. A phrase such an implementation may refer to one or more implementations and vice versa.

The word “exemplary” is used herein to mean “serving as an example or illustration.” Any aspect or design described herein as “exemplary” is not necessarily to be construed as preferred or advantageous over other aspects or designs.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

All structural and functional equivalents to the elements of the various aspects described throughout this disclosure that are known or later come to be known to those of ordinary skill in the art are expressly incorporated herein by reference and are intended to be encompassed by any claim. Furthermore, to the extent that the term “include,” “have,” or the like is used in the description, such term is intended to be inclusive in a manner similar to the term “comprise” as “comprise” is interpreted when employed as a transitional word.

What is claimed is:

1. An infusion device, the infusion device comprising:
a pump; and
a control unit configured by instructions that, when executed by a processor, cause the control unit to:
provide, using the pump, an intravenous infusion of a medication to a current patient;
display on a display screen, while the infusion is being provided by the pump, a representation of a status of the intravenous infusion;
send, during the infusion, to a remote records system, infusion information including a patient identifier and order identifier for the infusion currently being provided by the pump;
receive, from the remote records system, a confirmation of the infusion information including association information representative of an association between the infusion and the current patient or the pump;
receive, at the infusion device, an indication of a change to the infusion currently being provided by the pump;
compare the change with the association information received from the remote records system;
determine, based on comparing the change with the association information received from the remote records system, that the association received from the remote records system no longer applies to the changed infusion;
update the representation of the status displayed on the display screen to indicate that the association information received from the remote records system no longer applies to the intravenous infusion; and
send a message to the remote records system indicating that the infusion device has determined that the association information no longer applies to the changed infusion.

2. The infusion device of claim 1, wherein the representation of the status comprises a single colored light, and wherein updating the representation comprises changing a color of the single colored light from a first color to a second color.

3. The infusion device of claim 1, wherein the change to the infusion currently being provided by the infusion device comprises changing the medication to a new medication, and wherein determining that the association information received from the remote records system no longer applies to the changed infusion comprises determining, by the control unit of the infusion device, that a record for the current patient stored at the remote records system has not been updated with an order for the new medication.

4. The infusion device of claim 3, wherein sending the message to the remote records system comprises:
instructing the remote records system to stop recording infusion data pertaining to the medication and to start recording infusion data pertaining to the new medication.

5. The infusion device of claim 3, wherein sending the message to the remote records system comprises:
instructing the remote records system to buffer infusion data pertaining to the new medication until the changed infusion is confirmed at the remote records system,
wherein, on the changed infusion being confirmed at the remote records system, the remote records system creates an association between the changed infusion and the current patient or the infusion device, and begins recording data provided by the infusion device for the changed infusion in association with the current patient or the infusion device.

6. The infusion device of claim 1, wherein the change to the infusion currently being provided by the infusion device comprises changing the current patient to a new patient, and wherein determining that the association information received from the remote records system no longer applies to the changed infusion comprises determining, by the control unit of the infusion device, that the infusion device has not been assigned to the new patient at the remote records system, and
wherein sending the message to the remote records system comprises:
instructing the remote records system to disassociate the current patient from the infusion device.

7. The infusion device of claim 1, wherein the control unit is further configured to:
display on the display screen an indication that the remote records system is not synchronized with the changed infusion to include the association between the changed infusion and the current patient or the infusion device;
prompt for a confirmation to proceed with the changed infusion;
adjusting infusion parameters on the infusion device to implement the change to the infusion when the confirmation is received; and
terminating the infusion when the confirmation is not received.

8. The infusion device of claim 7, wherein the control unit is further configured to, when the confirmation is not received:
determine a second infusion being provided by the infusion device; and
terminating the second infusion.

9. A method, comprising:
displaying, on a display screen of an infusion device, while an intravenous infusion of a medication is being provided by the infusion device to a current patient, a representation of a status of the infusion;
sending, during the infusion, from the infusion device to a remote records system, infusion information including a patient identifier and order identifier for the infusion currently being provided by the infusion device;
receiving, from the remote records system, a confirmation of the infusion information including association information representative of an association between the infusion and the current patient or the infusion device;
receiving, at the infusion device, an indication of a change to the infusion currently being provided by the infusion device;
comparing the change with the association information received from the remote records system:
determining, based on comparing the change with the association information received from the remote records system, that the association information received from the remote records system no longer applies to the changed infusion;
updating the representation of the status displayed on the display screen to indicate that the association information received from the remote records system no longer applies to the intravenous infusion; and
sending a message to the remote records system indicating that the infusion device has determined that the association information no longer applies to the changed infusion.

10. The method of claim 9, wherein the representation of the status comprises a single colored light, and wherein updating the representation comprises changing a color of the single colored light from a first color to a second color.

11. The method of claim 9, wherein the change to the infusion currently being provided by the infusion device comprises changing the medication to a new medication, and wherein determining that the association information received from the remote records system no longer applies to the changed infusion comprises determining, by the infusion device, that a record for the current patient stored at the remote records system has not been updated with an order for the new medication.

12. The method of claim 11, wherein sending the message to the remote records system comprises:
instructing the remote records system to stop recording infusion data pertaining to the medication and to start recording infusion data pertaining to the new medication.

13. The method of claim 11, wherein sending the message to the remote records system comprises:
instructing the remote records system to buffer infusion data pertaining to the new medication until the remote records system until the changed infusion is confirmed at the remote records system,
wherein, on the changed infusion being confirmed at the remote records system, the remote records system creates an association between the changed infusion and the current patient or the infusion device, and begins recording data provided by the infusion device for the changed infusion in association with the current patient or the infusion device.

14. The method of claim 9, wherein the change to the infusion currently being provided by the infusion device comprises changing the current patient to a new patient, and wherein determining that the association information received from the remote records system no longer applies to the changed infusion comprises determining, by the infusion device, that the infusion device has not been assigned to the new patient at the remote records system, and wherein sending the message to the remote records system comprises:
instructing the remote records system to disassociate the current patient from the infusion device.

15. The method of claim 9, further comprising:
display on the display screen an indication that the remote records system is not synchronized with the changed infusion to include the association between the changed infusion and the current patient or the infusion device;
prompt for a confirmation to proceed with the changed infusion;
adjusting infusion parameters on the infusion device to implement the change to the infusion when the confirmation is received; and
terminating the infusion when the confirmation is not received.

16. The method of claim 15, further comprising, when the confirmation is not received:
determine a second infusion being provided by the infusion device; and
terminating the second infusion.

17. A method performed by a computing system, comprising:
receiving, from an infusion device, infusion information pertaining to an infusion of a medication being provided by the infusion device to a current patient, the infusion information including a patient identifier and order identifier for the infusion currently being provided by the infusion device;
determining an association between the infusion of the medication and the current patient or the infusion device;
providing, to the infusion device, a confirmation of the infusion information including association information representative of the determined association between the infusion and the current patient or the infusion device;
receiving, from the infusion device, an indication that the infusion device has determined that the association information no longer applies to the infusion or the current patient;
receiving, from the infusion device, in connection with the indication, new association information including a new patient or a new medication for the current patient;
automatically disassociating the current patient or the infusion from the infusion device; and
automatically initiate creation of a new association between the infusion device and the new patient or the new medication based on the new association information received from the infusion device.

18. The method of claim 17, wherein receiving the indication comprises receiving an indication that the medication being provided by the infusion device was changed to the new medication.

19. The method of claim 18, wherein receiving the indication that the infusion device has determined that the association information no longer applies to the infusion or the current patient comprises:
receiving an instruction to stop recording infusion data pertaining to the medication, the method further comprising:
initiating, remote from the infusion device, a recording of infusion data pertaining to the new medication.

20. The method of claim 18, further comprising:
buffering, remote from the infusion device, infusion data pertaining to the new medication until the creation of the new association is confirmed by the computing system,
recording, when the creation of a new association is confirmed, data provided by the infusion device for the new patient or the new medication for the current patient.
